(19) United States
(12) Patent Application Publication
(10) Pub. No.: US 2024/0285399 A1
(43) Pub. Date: Aug. 29, 2024

(54) COMMISSURE MARKER FOR A PROSTHETIC HEART VALVE

(71) Applicant: EDWARDS LIFESCIENCES CORPORATION, Irvine, CA (US)

(72) Inventors: Tyler Dale O'Dell, Irvine, CA (US); Swathi Ravi, Irvine, CA (US)

(21) Appl. No.: 18/652,967

(22) Filed: May 2, 2024

Related U.S. Application Data
(63) Continuation of application No. PCT/US2022/049429, filed on Nov. 9, 2022.
(60) Provisional application No. 63/278,597, filed on Nov. 12, 2021.

(51) Int. Cl. A61F 2/24; A61L 31/02; A61L 31/18

(52) U.S. Cl. CPC A61F 2/2418; A61L 31/022; A61L 31/18; A61F 2220/0075; A61F 2230/0013; A61F 2230/0019; A61F 2250/001; A61F 2250/0098

(57) ABSTRACT

Prosthetic heart valves including a radiopaque marker configured to indicate a location of a commissure of the prosthetic heart valve are disclosed. As one example, a prosthetic heart valve includes an annular frame comprising a plurality of struts, a plurality of leaflets arranged within the frame, and at least one commissure comprising commissure tabs of two adjacent leaflets of the plurality of leaflets connected to each other, the at least one commissure coupled to the frame. The valve further includes a radiopaque marker, separate from the frame, attached to the commissure, where the marker is configured to indicate a location of the commissure of the prosthetic heart valve.

### CROSS-REFERENCE TO RELATED APPLICATIONS

This application is a continuation of PCT Application No. PCT/US2022/049429, filed Nov. 9, 2022, which claims the benefit of U.S. Provisional Patent Application No. 63/278,597, filed Nov. 12, 2021, each of which is incorporated by reference herein in its entirety.

### FIELD

The present disclosure relates to prosthetic heart valves and markers for prosthetic heart valves configured to indicate a location of a commissure of the prosthetic heart valve.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery and the aorta) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery apparatus so that the prosthetic valve can self-expand to its functional size.

### SUMMARY

Described herein are prosthetic heart valves, delivery apparatuses, and methods for implanting prosthetic heart valves. The disclosed prosthetic heart valves, delivery apparatuses, and methods can, for example, provide visualization of a location of a commissures of the prosthetic heart valve relative to the native anatomy or a previously implanted prosthetic heart valve. As such, the devices and methods disclosed herein can, among other things, overcome one or more of the deficiencies of typical prosthetic heart valves and their delivery apparatuses.

A prosthetic heart valve can comprise a frame and a valve structure coupled to the frame. In addition to these components, a prosthetic heart valve can further comprise one or more of the components disclosed herein.

In some examples, a prosthetic heart valve can comprise a sealing member configured to reduce paravalvular leakage.

In some examples, a prosthetic heart valve can comprise a frame, a plurality of leaflets arranged within the frame, at least on commissure comprising commissure tabs of two adjacent leaflets of the plurality of leaflets connected to each other, the at least one commissure coupled to the frame, and at least one radiopaque marker attached to the commissure.

In some examples, a prosthetic heart valve comprises an annular frame comprising a plurality of struts, a plurality of leaflets arranged within the frame, and at least one commissure comprising commissure tabs of two adjacent leaflets of the plurality of leaflets connected to each other, the at least one commissure coupled to the frame. The prosthetic heart valve further comprises at least one radiopaque marker, separate from the frame, attached to the commissure, where the marker is configured to indicate a location of the commissure of the prosthetic heart valve.

In some examples, a prosthetic heart valve comprises a frame comprising an inflow end, an outflow end, and a plurality of support posts including a commissure window; a plurality of leaflets arranged within the frame and configured to regulate a flow of blood through the frame in one direction between the inflow end and outflow end; at least one commissure comprising commissure tabs of two adjacent leaflets of the plurality of leaflets connected to each other and extending radially through the commissure window of a corresponding support post of the plurality of support posts, the at least one commissure connected to the corresponding support post; and a radiopaque marker attached to the commissure. The marker is configured to indicate a location of the commissure of the prosthetic heart valve.

In some examples, a prosthetic heart valve comprises a frame, the frame comprising a plurality of support posts spaced apart about a circumference of the frame, each support post of the plurality of support posts including a commissure window; and one or more expansion and locking mechanisms that are configured to radially expand and/or compress the frame, each of the one or more expansion and locking mechanism comprising a pair of axially aligned posts and a threaded rod extending through the pair of posts. The prosthetic heart valve further comprises a plurality of leaflets arranged within the frame and configured to regulate a flow of blood through the frame in one direction; at least one commissure comprising commissure tabs of two adjacent leaflets of the plurality of leaflets connected to each other and extending radially through the commissure window of a corresponding support post, the at least one commissure connected to the corresponding support post; and a radiopaque marker attached to the commissure.

In some examples, a prosthetic heart valve comprises one or more of the components recited in Examples 1-51 below.

A method for implanting a prosthetic heart valve can comprise at a native valve of a heart, visualizing under fluoroscopy a position of a radiopaque marker attached to a commissure of a prosthetic heart valve relative to the native valve, and implanting the prosthetic heart valve into the native valve or a previously implanted prosthetic heart valve such that the commissure of the prosthetic heart valve to which the marker is attached is aligned with the native commissure of the native valve or a selected commissure of the previously implanted prosthetic heart valve.

In some examples, a method comprises at a native valve of a heart, visualizing under fluoroscopy a position of a radiopaque marker attached to a commissure of a prosthetic heart valve relative to the native valve, wherein the commissure comprises commissure tabs of two adjacent leaflets of a plurality of leaflets of the prosthetic heart valve connected to each other, the commissure connected to a frame of the prosthetic heart valve, and wherein the marker is separate from the frame. The method further comprises implanting the prosthetic heart valve into the native valve or a previously implanted prosthetic heart valve such that the commissure of the prosthetic heart valve to which the marker is attached is aligned with the native commissure of the native valve or a selected commissure of the previously implanted prosthetic heart valve.

In some examples, a method comprises one or more of the features recited in Examples 52-60 below.

The above methods can be performed on a living animal or on a simulation, such as on a cadaver, cadaver heart, anthropomorphic ghost, simulator (e.g., with body parts, heart, tissue, etc. being simulated).

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1A is a perspective view of one example of a prosthetic valve including a frame and a plurality of leaflets attached to the frame.

FIG. 1B is a perspective view of the prosthetic valve of FIG. 1A with an outer skirt disposed around the frame.

FIG. 2A is a perspective view of a frame for the prosthetic valve of FIG. 1A.

FIG. 2B is a front portion of the frame shown in FIG. 2A.

FIG. 3 is a side elevation view of a delivery apparatus for a prosthetic device, such as a prosthetic valve, according to one example.

FIG. 4 is a schematic of an exemplary heart showing a position of coronary arteries relative to an aortic valve.

FIG. 5A is a cross-sectional view of an aortic valve illustrating a first positioning of a prosthetic valve within the aortic valve where commissures of the prosthetic valve at least partially block one or more openings to the coronary arteries.

FIG. 5B is a cross-sectional view of an aortic valve illustrating a second positioning of a prosthetic valve within the aortic valve where commissures of the prosthetic valve are circumferentially aligned with native commissure of the aortic valve, thereby maintaining access to the coronary arteries.

FIG. 6 is a perspective view of the prosthetic valve of FIG. 1A with an exemplary radiopaque marker attached to a commissure of the prosthetic valve.

FIG. 7 is a schematic illustration of a portion of a process for forming a commissure of a prosthetic heart valve.

FIG. 8 is a schematic illustration of another portion of the process for forming the commissure of the prosthetic heart valve.

FIGS. 9A-9C illustrate exemplary locations for a radiopaque marker at a commissure of a prosthetic heart valve.

FIG. 10 illustrates an exemplary radiopaque marker configured to be attached to a commissure of a prosthetic heart valve.

FIG. 11 illustrates an exemplary radiopaque marker configured to be attached to a commissure of a prosthetic heart valve.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

As used herein, “e.g.” means “for example,” and “i.e.” means “that is.”

### Overview of the Disclosed Technology

Prosthetic valves disclosed herein can be radially compressible and expandable between a radially compressed state and a radially expanded state. Thus, the prosthetic valves can be crimped on or retained by an implant delivery apparatus in the radially compressed state while being advanced through a patient's vasculature on the delivery apparatus. The prosthetic valve can be expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

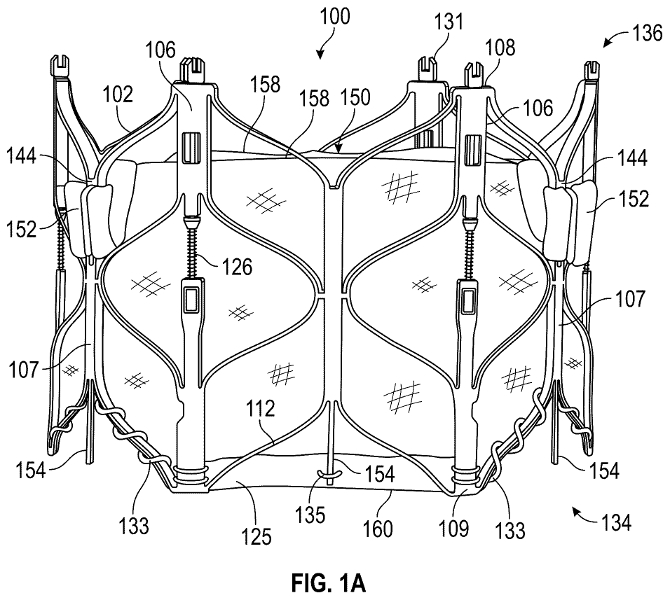

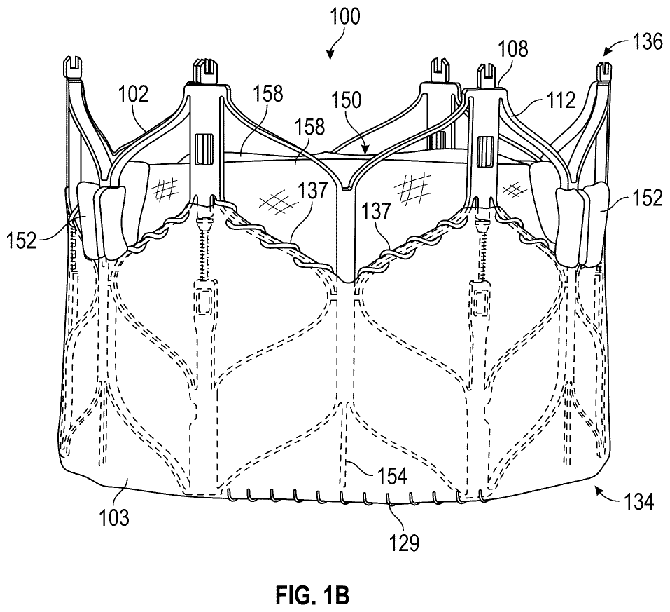

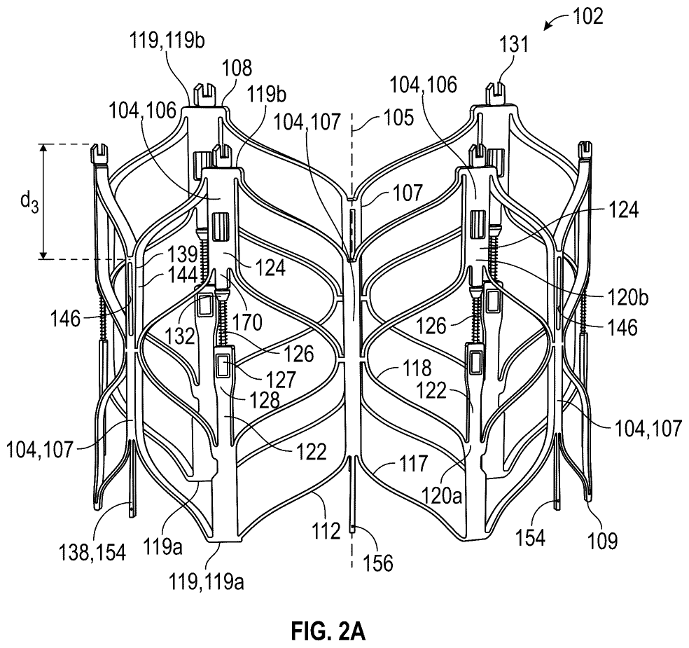

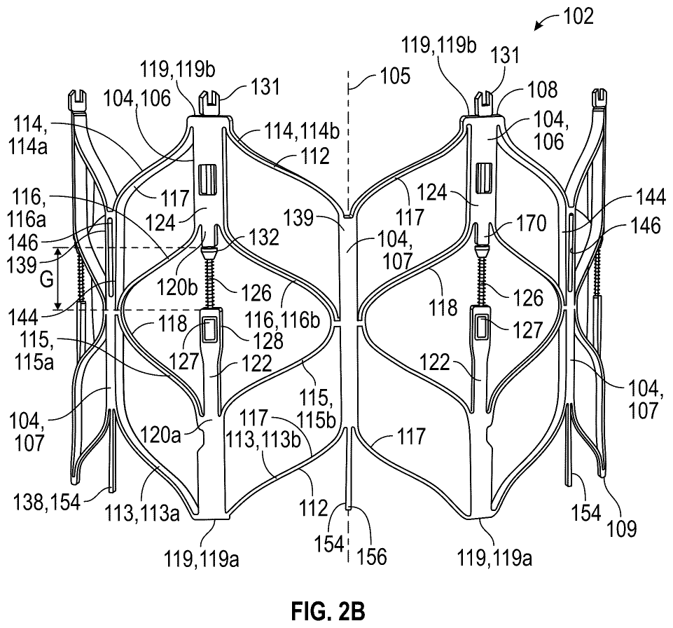

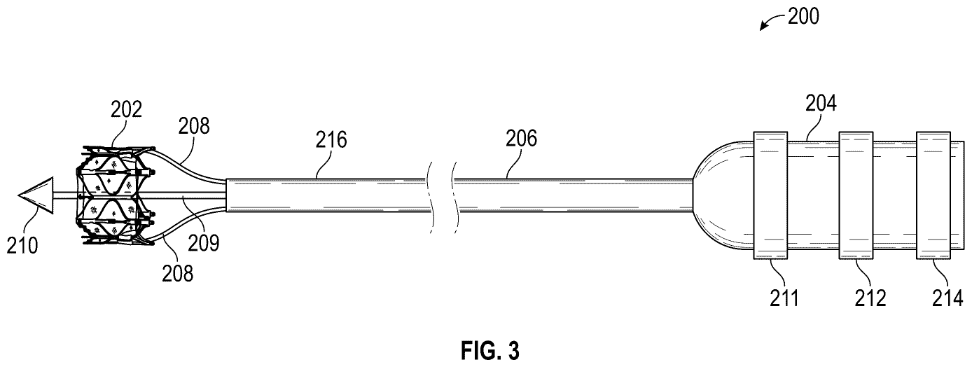

FIGS. 1A-2B illustrate an exemplary prosthetic device (e.g., prosthetic heart valve) that can be advanced through a patient's vasculature, such as to a native heart valve, by a delivery apparatus, such as the exemplary delivery apparatus shown in FIG. 3. The frame of the prosthetic heart valve can include one or more mechanical expansion and locking mechanisms that can be integrated into the frame-specifically, into axially extending posts of the frame. The mechanical expansion and/or locking mechanisms can be removably coupled to, and/or actuated by, the delivery apparatus to radially expand the prosthetic heart valve and lock the prosthetic heart valve in one or more radially expanded states.

When deploying the prosthetic valve at the native heart valve, the radially expanded prosthetic valve typically is deployed at a random radial orientation relative to the native valve. As such, in some implantations, one of the commissures of the prosthetic valve may be arranged in front of (e.g., adjacent to) a coronary ostium of the aorta. This arrangement may reduce coronary access (e.g., blood flow to the coronary arteries from the aorta) and/or create difficulties during future cardiovascular interventions that aim to maintain or increase coronary access. Further, after implanting a prosthetic heart valve, it may be desirable to confirm a location of the commissures of the prosthetic heart valve relative to the commissures of the native heart valve and/or visualize a location of the commissures of the implanted prosthetic heart valve relative to the commissures of a subsequently implanted prosthetic valve during a valve-in-valve procedure.

Accordingly, a need exists for improved prosthetic heart valve configurations that allow for identification of a position of one or more commissures of the prosthetic heart valve during an implantation procedure and/or after implantation at a native heart valve.

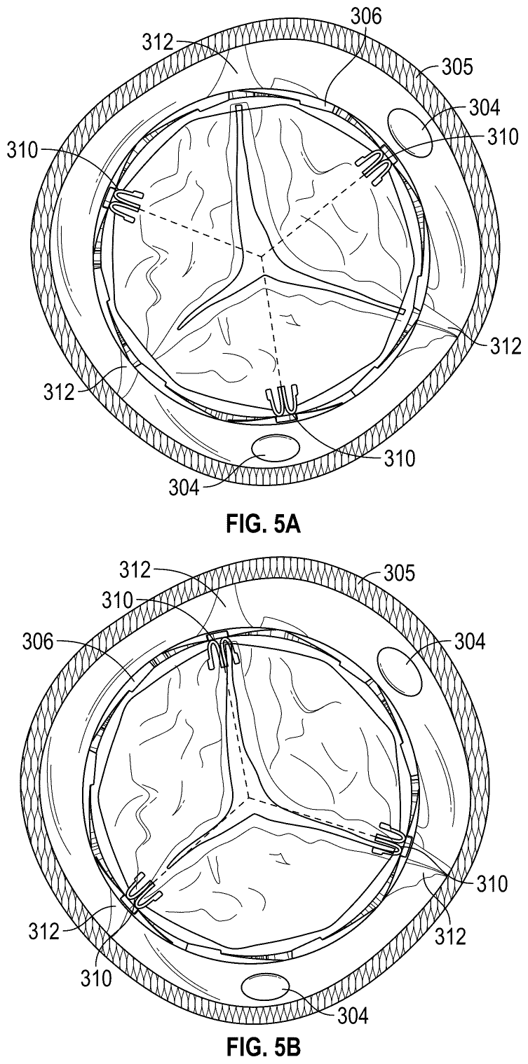

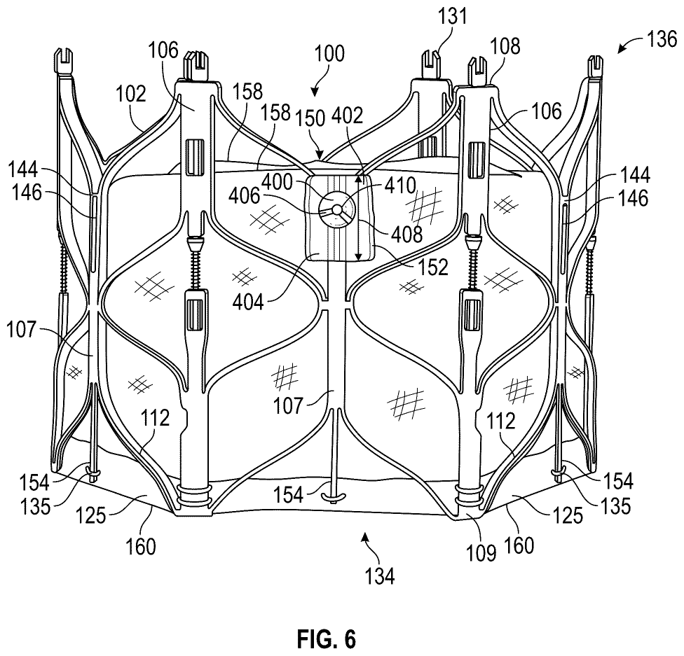

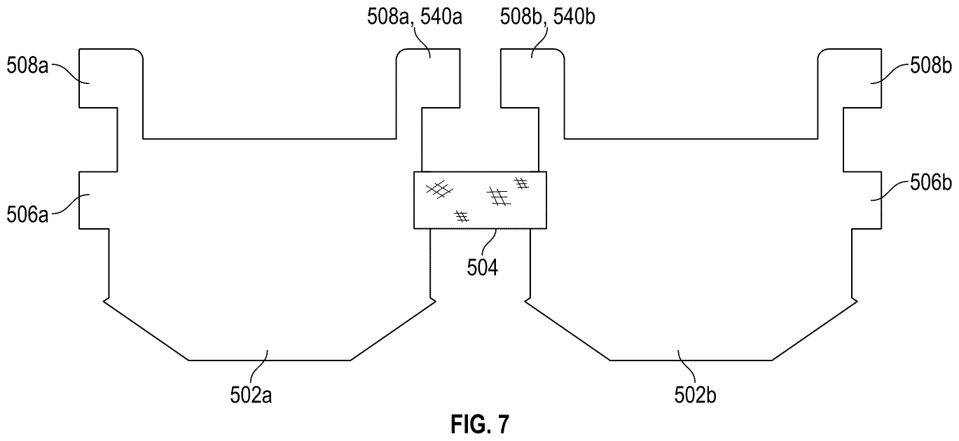

Described herein are examples of prosthetic valve delivery apparatuses and methods for delivering and implanting a radially expandable prosthetic valve at a native valve of a heart such that commissures of the prosthetic valve are in a specified circumferential orientation relative to commissures of the native valve or a previously implanted prosthetic heart valve. In some examples, as shown at FIG. 5B, the specified circumferential orientation includes the prosthetic valve commissures being aligned with the native valve commissures or the previously implanted prosthetic heart valve commissures. FIGS. 6 and 9A-11 show examples of radiopaque markers and different locations for the radiopaque markers on the commissures of the prosthetic valve which can enable the prosthetic valve to be visualized during an implantation procedure. As such, a prosthetic valve including such radiopaque markers can be more easily aligned with the native anatomy or a previously implanted prosthetic valve in the specified circumferential orientation.

### Examples of the Disclosed Technology

FIGS. 1A-2B show an exemplary prosthetic valve 100, according to one example. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO2020/247907, which is incorporated herein by reference. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

FIGS. 1A-2B illustrate an exemplary prosthetic valve 100 (which also may be referred to herein as “prosthetic heart valve 100”) having a frame 102. FIGS. 2A-2B show the frame 102 by itself, while FIGS. 1A-1B show the frame 102 with a valvular structure 150 (which can comprise leaflets 158, as described further below) mounted within and to the annular frame 102. FIG. 1B additionally shows an optional skirt assembly comprising an outer skirt 103. While only one side of the frame 102 is depicted in FIG. 2B, it should be appreciated that the frame 102 forms an annular structure having an opposite side that is substantially identical to the portion shown in FIG. 1B, as shown in FIGS. 1A-2A.

As shown in FIGS. 1A and 1B, the valvular structure 150 is coupled to and supported inside the frame 102. The valvular structure 150 is configured to regulate the flow of blood through the prosthetic valve 100, from an inflow end portion 134 to an outflow end portion 136. The valvular structure 150 can include, for example, a leaflet assembly comprising one or more leaflets 158 made of flexible material. The leaflets 158 can be made from in whole or part, biological material, bio-compatible synthetic materials, or other such materials. Suitable biological material can include, for example, bovine pericardium (or pericardium from other sources). The leaflets 158 can be secured to one another at their adjacent sides to form commissures 152, each of which can be secured to a respective commissure support structure 144 (also referred to herein as “commissure supports”) and/or to other portions of the frame 102, as described in greater detail below.

In the example depicted in FIGS. 1A and 1B, the valvular structure 150 includes three leaflets 158, which can be arranged to collapse in a tricuspid arrangement. Each leaflet 158 can have an inflow edge portion 160 (which can also be referred to as a cusp edge portion) (FIG. 1A). The inflow edge portions 160 of the leaflets 158 can define an undulating, curved scallop edge that generally follows or tracks portions of struts 112 of frame 102 in a circumferential direction when the frame 102 is in the radially expanded configuration. The inflow edge portions 160 of the leaflets 158 can be referred to as a “scallop line.”

The prosthetic valve 100 may include one or more skirts mounted around the frame 102. For example, as shown in FIG. 1B, the prosthetic valve 100 may include an outer skirt 103 mounted around an outer surface of the frame 102. The outer skirt 103 can function as a scaling member for the prosthetic valve 100 by sealing against the tissue of the native valve annulus and helping to reduce paravalvular leakage past the prosthetic valve 100. In some cases, an inner skirt (not shown) may be mounted around an inner surface of the frame 102. The inner skirt can function as a sealing member to prevent or decrease perivalvular leakage, to anchor the leaflets 158 to the frame 102, and/or to protect the leaflets 158 against damage caused by contact with the frame 102 during crimping and during working cycles of the prosthetic valve 100. In some examples, the inflow edge portions 160 of the leaflets 158 can be sutured to the inner skirt generally along the scallop line. The inner skirt can in turn be sutured to adjacent struts 112 of the frame 102. In some examples, as shown in FIG. 1A, the leaflets 158 can be sutured directly to the frame 102 or to a reinforcing strip 125 (e.g., a fabric strip) which is then sutured to the frame 102, along the scallop line via stitches (e.g., whip stitches) 133.

The inner and outer skirts and the reinforcing strip 125 can be formed from any of various suitable biocompatible materials, including any of various synthetic materials, including fabrics (e.g., polyethylene terephthalate fabric) or natural tissue (e.g., pericardial tissue). Further details regarding the use of skirts or sealing members in prosthetic valve can be found, for example, in U.S. Patent Publication No. 2020/0352711, which is incorporated herein by reference.

Further details regarding the assembly of the leaflet assembly and the assembly of the leaflets and the skirts to the frame can be found, for example, in U.S. Provisional Application Nos. 63/209,904, filed Jun. 11, 2021, and 63/224,534, filed Jul. 22, 2021, which are incorporated herein by reference. Further details of the construction and function of the frame 102 can be found in International Patent Application No. PCT/US2021/052745, filed Sep. 30, 2021, which is incorporated herein by reference.

The frame 102, which is shown alone and in greater detail in FIGS. 2A and 2B, comprises and inflow end 109, an outflow end 108, and a plurality of axially extending posts 104. The axial direction of the frame 102 is indicated by a longitudinal axis 105, which extends from the inflow end 109 to the outflow end 108 (FIGS. 2A and 2B). Some of the posts 104 can be arranged in pairs of axially aligned first and second struts or posts 122, 124. An actuator 126 (such as the illustrated threaded rod or bolt) can extend through one or more pairs of posts 122, 124 to form an integral expansion and locking mechanism or actuator mechanism 106 configured to radially expand and compress the frame 102, as further described below. One or more of posts 104 can be configured as support posts 107.

The actuator mechanisms 106 (which can be used to radially expand and/or radially compress the prosthetic valve 100) can be integrated into the frame 102 of the prosthetic valve 100, thereby reducing the crimp profile and/or bulk of the prosthetic valve 100. Integrating the actuator mechanisms 106 (which can also be referred to herein as “expansion and locking mechanisms”) into the frame 102 can also simplify the design of the prosthetic valve 100, making the prosthetic valve 100 less costly and/or easier to manufacture. In the illustrated example, an actuator 126 extends through each pair of axially aligned posts 122, 124. In some examples, one or more of the pairs of posts 122, 124 can be without a corresponding actuator.

The posts 104 can be coupled together by a plurality of circumferentially extending link members or struts 112. Each strut 112 extends circumferentially between adjacent posts 104 to connect all of the axially extending posts 104. As one example, the prosthetic valve 100 can include equal numbers of support posts 107 and pairs of actuator posts 122, 124 and the pairs of posts 122, 124 and the support posts 107 can be arranged in an alternating order such that each strut 112 is positioned between one of the pairs of posts 122, 124 and one of the support posts 107 (i.e., each strut 112 can be coupled on one end to one of the posts 122, 124 and can be coupled on the other end to one of the support posts 107). However, the prosthetic valve 100 can include different numbers of support posts 107 and pairs of posts 122, 124 and/or the pairs of posts 122, 124 and the support posts 107 can be arranged in a non-alternating order, in some examples.

As illustrated in FIG. 2B, the struts 112 can include a first row of struts 113 at or near the inflow end 109 of the prosthetic valve 100, a second row of struts 114 at or near the outflow end 108 of the prosthetic valve 100, and third and fourth rows of struts 115, 116, respectively, positioned axially between the first and second rows of struts 113, 114. The struts 112 can form and/or define a plurality of cells (i.e., openings) in the frame 102. For example, the struts 113, 114, 115, and 116 can at least partially form and/or define a plurality of first cells 117 and a plurality of second cells 118 that extend circumferentially around the frame 102. Specifically, each first cell 117 can be formed by two struts 113a, 113b of the first row of struts 113, two struts 114a, 114b of the second row of struts 114, and two of the support posts 107. Each second cell 118 can be formed by two struts 115a, 115b of the third row of struts 115 and two struts 116a, 116b of the fourth row of struts 116. As illustrated in FIGS. 2A and 2B, each second cell 118 can be disposed within one of the first cells 117 (i.e., the struts 115a-116b forming the second cells 118 are disposed between the struts forming the first cells 117 (i.e., the struts 113a, 113b and the struts 114a, 114b), closer to an axial midline of the frame 102 than the struts 113a-114b).

As illustrated in FIGS. 2A and 2B, the struts 112 of frame 102 can comprise a curved shape. Each first cell 117 can have an axially-extending hexagonal shape including first and second apices 119 (e.g., an inflow apex 119a and an outflow apex 119b). In examples where the delivery apparatus is releasably connected to the outflow apices 119b (as described below), each inflow apex 119a can be referred to as a “distal apex” and each outflow apex 119b can be referred to as a “proximal apex”. Each second cell 118 can have a diamond shape including first and second apices 120 (e.g., distal apex 120a and proximal apex 120b). In some examples, the frame 102 comprises six first cells 117 extending circumferentially in a row, six second cells 118 extending circumferentially in a row within the six first cells 117, and twelve posts 104. However, in some examples, the frame 102 can comprise a greater or fewer number of first cells 117 and a correspondingly greater or fewer number of second cells 118 and posts 104.

As noted above, some of the posts 104 can be arranged in pairs of first and second posts 122, 124. The posts 122, 124 are aligned with each other along the length of the frame 102 and are axially separated from one another by a gap G (FIG. 2B) (those with actuators 126 can be referred to as actuator posts or actuator struts). Each first post 122 (i.e., the lower post shown in FIGS. 2A and 2B) can extend axially from the inflow end 109 of the prosthetic valve 100 toward the second post 124, and the second post 124 (i.e., the upper post shown in FIGS. 2A and 2B) can extend axially from the outflow end 108 of the prosthetic valve 100 toward the first post 122. For example, each first post 122 can be connected to and extend from an inflow apex 119a and each second post 124 can be connected to and extend from an outflow apex 119b. Each first post 122 and the second post 124 can include an inner bore configured to receive a portion of an actuator member, such as in the form of a substantially straight threaded rod 126 (or bolt) as shown in the illustrated example. The threaded rod 126 also may be referred to herein as actuator 126, actuator member 126, and/or screw actuator 126. In examples where the delivery apparatus can be releasably connected to the outflow end 108 of the frame 102, the first posts 122 can be referred to as distal posts or distal axial struts and the second posts 124 can be referred to as proximal posts or proximal axial struts.

Each threaded rod 126 extends axially through a corresponding first post 122 and second post 124. Each threaded rod 126 also extends through a bore of a nut 127 captured within a slot or window formed in an end portion 128 of the first post 122. The threaded rod 126 has external threads that engage internal threads of the bore of the nut 127. The inner bore of the second post 124 (through which the threaded rod 126 extends) can have a smooth and/or non-threaded inner surface to allow the threaded rod 126 to slide freely within the bore. Rotation of the threaded rod 126 relative to the nut 127 produces radial expansion and compression of the frame 102, as further described below.

In some examples, the threaded rod 126 can extend past the nut 127 toward the inflow end 109 of the frame 102 into the inner bore of the first post 122. The nut 127 can be held in a fixed position relative to the first post 122 such that the nut 127 does not rotate relative to the first post 122. In this way, whenever the threaded rod 126 is rotated (e.g., by a physician) the threaded rod 126 can rotate relative to both the nut 127 and the first post 122. The engagement of the external threads of the threaded rod 126 and the internal threads of the nut 127 prevent the rod 126 from moving axially relative to the nut 127 and the first post 122 unless the threaded rod 126 is rotated relative to the nut 127. Thus, the threaded rod 126 can be retained or held by the nut 127 and can only be moved relative to the nut 127 and/or the first post 122 by rotating the threaded rod 126 relative to the nut 127 and/or the first post 122. In some examples, in lieu of using the nut 127, at least a portion of the inner bore of the first post 122 can be threaded. For example, the bore along the end portion 128 of the first post 122 can comprise inner threads that engage the external threaded rod 126 such that rotation of the threaded rod causes the threaded rod 126 to move axially relative to the first post 122.

When a threaded rod 126 extends through and/or is otherwise coupled to a pair of axially aligned posts 122, 124, the pair of axially aligned posts 122, 124 and the threaded rod 126 can serve as one of the expansion and locking mechanisms 106. In some examples, a threaded rod 126 can extend through each pair of axially aligned posts 122, 124 so that all of the posts 122, 124 (with their corresponding rods 126) serve as expansion and locking mechanisms 106. As just one example, the prosthetic valve 100 can include six pairs of posts 122, 124, and each of the six pairs of posts 122, 124 with their corresponding rods 126 can be configured as one of the expansion and locking mechanisms 106 for a total of six expansion and locking mechanisms 106. In some examples, not all pairs of posts 122, 124 need be expansion and locking mechanisms (i.e., actuators). If a pair of posts 122, 124 is not used as an expansion and locking mechanism, a threaded rod 126 need not extend through the posts 122, 124 of that pair.

The threaded rod 126 can be rotated relative to the nut 127, the first post 122, and the second post 124 to axially foreshorten and/or axially elongate the frame 102, thereby radially expanding and/or radially compressing, respectively, the frame 102 (and therefore the prosthetic valve 100). Specifically, when the threaded rod 126 is rotated relative to the nut 127, the first post 122, and the second post 124, the first and second posts 122, 124 can move axially relative to one another, thereby widening or narrowing the gap G (FIG. 2B) separating the posts 122, 124, and thereby radially compressing or radially expanding the prosthetic valve 100, respectively. Thus, the gap G (FIG. 2B) between the first and second posts 122, 124 narrows as the frame 102 is radially expanded and widens as the frame 102 is radially compressed.

The threaded rod 126 can extend proximally past the proximal end of the second post 124 and can include a head portion 131 at its proximal end that can serve at least two functions. First, the head portion 131 can removably or releasably couple the threaded rod 126 to a respective actuator assembly of a delivery apparatus that can be used to radially expand and/or radially compress the prosthetic valve 100 (e.g., the delivery apparatus 200 of FIG. 3, as described below). Second, the head portion 131 can prevent the second post 124 from moving proximally relative to the threaded rod 126 and can apply a distally directed force to the second post 124, such as when radially expanding the prosthetic valve 100. Specifically, the head portion 131 can have a width greater than a diameter of the inner bore of the second post 124 such that the head portion 131 is prevented from moving into the inner bore of the second post 124. Thus, as the threaded rod 126 is threaded farther into the nut 127, the head portion 131 of the threaded rod 126 draws closer to the nut 127 and the first post 122, thereby drawing the second post 124 towards the first post 122, and thereby axially foreshortening and radially expanding the prosthetic valve 100.

The threaded rod 126 also can include a stopper 132 (e.g., in the form of a nut, washer or flange) disposed thereon. The stopper 132 can be disposed on the threaded rod 126 such that it sits within the gap G. Further, the stopper 132 can be integrally formed on or fixedly coupled to the threaded rod 126 such that it does not move relative to the threaded rod 126. Thus, the stopper 132 can remain in a fixed axial position on the threaded rod 126 such that it moves in lockstep with the threaded rod 126.

Rotation of the threaded rod 126 in a first direction (e.g., clockwise) can cause corresponding axial movement of the first and second posts 122, 124 toward one another, thereby decreasing the gap G and radially expanding the frame 102, while rotation of the threaded rod 126 in an opposite second direction causes corresponding axial movement of the first and second posts 122, 124 away from one another, thereby increasing the gap G and radially compressing the frame. When the threaded rod 126 is rotated in the first direction, the head portion 131 of the rod 126 bears against an adjacent surface of the frame (e.g., an outflow apex 119b), while the nut 127 and the first post 122 travel proximally along the threaded rod 126 toward the second post 124, thereby radially expanding the frame. As the frame 102 moves from a compressed configuration to an expanded configuration, the gap G between the first and second posts 122, 124 can narrow.

When the threaded rod 126 is rotated in the second direction, the threaded rod 126 and the stopper 132 move toward the outflow end 110 of the frame until the stopper 132 abuts the inflow end 170 of the second post 124 (as shown in FIGS. 2A and 2B). Upon further rotation of the rod 126 in the second direction, the stopper 132 can apply a proximally directed force to the second post 124 to radially compress the frame 102. Specifically, during crimping/radial compression of the prosthetic valve 100, the threaded rod 126 can be rotated in the second direction (e.g., counterclockwise) causing the stopper 132 to push against (i.e., provide a proximally directed force to) the inflow end 170 of the second post 124, thereby causing the second post 124 to move away from the first post 122, and thereby axially elongating and radially compressing the prosthetic valve 100.

Thus, each of the second posts 124 can slide axially relative to a corresponding one of the first posts 122 but can be axially retained and/or restrained between the head portion 131 of a threaded rod 126 and a stopper 132. That is, each second post 124 can be restrained at its proximal end by the head portion 131 of the threaded rod 126 and at its distal end by the stopper 132. In this way, the head portion 131 can apply a distally directed force to the second post 124 to radially expand the prosthetic valve 100 while the stopper 132 can apply a proximally directed force to the second post 124 to radially compress the prosthetic valve 100. As explained above, radially expanding the prosthetic valve 100 axially foreshortens the prosthetic valve 100, causing an inflow end portion 134 and outflow end portion 136 of the prosthetic valve 100 (FIGS. 1A and 1B) to move towards one another axially, while radially compressing the prosthetic valve 100 axially elongates the prosthetic valve 100, causing the inflow and outflow end portions 134, 136 to move away from one another axially.

In some examples, the threaded rod 126 can be fixed against axial movement relative to the second post 124 (and the stopper 132 can be omitted) such that rotation of the threaded rod 126 in the first direction produces proximal movement of the nut 127 and radial expansion of the frame 102 and rotation of the threaded rod 126 in the second direction produces distal movement of the nut 127 and radial compression of the frame 102.

As also introduced above, some of the posts 104 can be configured as support posts 107. As shown in FIGS. 2A and 2B, the support posts 107 can extend axially between the inflow and outflow ends 109, 108 of the frame 102 and each can have an inflow end portion 138 and an outflow end portion 139. The outflow end portion 139 of one or more support posts 107 can include a commissure support structure or member 144. The commissure support structure 144 can comprise strut portions defining a commissure opening 146 therein.

The commissure opening 146 (which can also be referred to herein as a “commissure window 146”) can extend radially through a thickness of the support post 107 and can be configured to accept a portion of a valvular structure 150 (e.g., a commissure 152) to couple the valvular structure 150 to the frame 102. For example, each commissure 152 can be mounted to a respective commissure support structure 144, such as by inserting a pair of commissure tabs of adjacent leaflets 158 through the commissure opening 146 and suturing the commissure tabs to each other and/or the commissure support structure 144. In some examples, the commissure opening 146 can be fully enclosed by the support post 107 such that a portion of the valvular structure 150 can be slid radially through the commissure opening 146, from an interior to an exterior of the frame 102, during assembly. In the illustrated example, the commissure opening 146 has a substantially rectangular shape that is shaped and sized to receive commissure tabs of two adjacent leaflets therethrough. However, in some examples, the commissure opening can have any of various shapes (e.g., square, oval, square-oval, triangular, L-shaped, T-shaped, C-shaped, etc.).

The commissure openings 146 are spaced apart about the circumference of frame 102 (or angularly spaced apart about frame 102). The spacing may or may not be even. In one example, the commissure openings 146 are axially offset from the outflow end 108 of the frame 102 by an offset distance d₃ (indicated in FIG. 2A). As an example, the offset distance d₃ may be in a range from 2 mm to 6 mm. In general, the offset distance d₃ should be selected such that when the leaflets are attached to the frame 102 via the commissure openings 146, the free edge portions (e.g., outflow edge portions) of the leaflets 158 will not protrude from or past the outflow end 108 of the frame 102.

The frame 102 can comprise any number of support posts 107, any number of which can be configured as commissure support structures 144. For example, the frame 102 can comprise six support posts 107, three of which are configured as commissure support structures 144. However, in some examples, the frame 102 can comprise more or less than six support posts 107 and/or more or less than three commissure support structures 144.

The inflow end portion 138 of each support post 107 can comprise an extension 154 (show as a cantilevered strut in FIGS. 2A and 2B) that extends toward the inflow end 109 of the frame 102. Each extension 154 can comprise an aperture 156 extending radially through a thickness of the extension 154. In some examples, the extension 154 can extend such that an inflow edge of the extension 154 aligns with or substantially aligns with the inflow end 109 of the frame 102. In use, the extension 154 can prevent or mitigate portions of an outer skirt from extending radially inwardly and thereby prevent or mitigate any obstruction of flow through the frame 102 caused by the outer skirt. The extensions 154 can further serve as supports to which portions of the inner and/or outer skirts and/or the leaflets and/or the reinforcing skirt 125 can be coupled. For example, sutures used to connect the inner and/or outer skirts and/or the leaflets and/or the reinforcing skirt 125 can be wrapped around the extensions 154 and/or can extend through apertures 156.

As an example, each extension 154 can have an aperture 156 (FIG. 2A) or other features to receive a suture or other attachment material for connecting an adjacent inflow edge portion 160 of a leaflet 158 (FIG. 1A), the outer skirt 103 (in FIG. 1B), the reinforcing skirt 125, and/or an inner skirt. In some examples, the inflow edge portion 160 of each leaflet 158 can be connected to a corresponding extension via a suture 135 (FIG. 1A).

In some examples, the outer skirt 103 can be mounted around the outer surface of frame 102 as shown in FIG. 1B and the inflow edge of the outer skirt 103 (lower edge in FIG. 1B) can be attached to the reinforcing skirt 125 and/or the inflow edge portions 160 of the leaflets 158 that have already been secured to frame 102 as well as to the extensions 154 of the frame by sutures 129. The outflow edge of the outer skirt 103 (the upper edge in FIG. 1B) can be attached to selected struts with stitches 137. In implementations where the prosthetic valve includes an inner skirt, the inflow edge of the inner skirt can be secured to the inflow edge portions 160 before securing the cusp edge portions to the frame so that the inner skirt will be between the leaflets and the inner surface of the frame. After the inner skirt and leaflets are secured in place, then the outer skirt can be mounted around the frame as described above.

The frame 102 can be a unitary and/or fastener-free frame that can be constructed from a single piece of material (e.g., Nitinol, stainless steel or a cobalt-chromium alloy), such as in the form of a tube. The plurality of cells can be formed by removing portions (e.g., via laser cutting) of the single piece of material. The threaded rods 126 can be separately formed and then be inserted through the bores in the second (proximal) posts 124 and threaded into the threaded nuts 127.

In some examples, the frame 102 can be formed from a plastically-expandable material, such as stainless steel or a cobalt-chromium alloy. When the frame is formed from a plastically-expandable material, the prosthetic valve 100 can be placed in a radially compressed state along the distal end portion of a delivery apparatus for insertion into a patient's body. When at the desired implantation site, the frame 102 (and therefore the prosthetic valve 100) can be radially expanded from the radially compressed state to a radially expanded state via actuation of actuation assemblies of the delivery apparatus (as further described below), which rotate the rods 126 to produce expansion of the frame 102. During delivery to the implantation site, the prosthetic valve 100 can be placed inside of a delivery capsule (sheath) to protect against the prosthetic valve contacting the patient's vasculature, such as when the prosthetic valve is advanced through a femoral artery. The capsule can also retain the prosthetic valve in a compressed state having a slightly smaller diameter and crimp profile than may be otherwise possible without a capsule by preventing any recoil (expansion) of the frame once it is crimped onto the delivery apparatus.

In some examples, the frame 102 can be formed from a self-expandable material (e.g., Nitinol). When the frame 102 is formed from a self-expandable material, the prosthetic valve can be radially compressed and placed inside the capsule of the delivery apparatus to maintain the prosthetic valve in the radially compressed state while it is being delivered to the implantation site. When at the desired implantation site, the prosthetic valve is deployed or released from the capsule. In some examples, the frame (and therefore the prosthetic valve) can partially self-expand from the radially compressed state to a partially radially expanded state. The frame 102 (and therefore the prosthetic valve 100) can be further radially expanded from the partially expanded state to a further radially expanded state via actuation of actuation assemblies of the delivery apparatus (as further described below), which rotate the rods 126 to produce expansion of the frame.

As introduced above, the threaded rods 126 can removably couple the prosthetic valve 100 to actuator assemblies of a delivery apparatus. Referring to FIG. 3, it illustrates an exemplary delivery apparatus 200 for delivering a prosthetic device or valve 202 (e.g., prosthetic valve 100) to a desired implantation location. The prosthetic valve 202 can be releasably coupled to the delivery apparatus 200. It should be understood that the delivery apparatus 200 and other delivery apparatuses disclosed herein can be used to implant prosthetic devices other than prosthetic valves, such as stents or grafts.

The delivery apparatus 200 in the illustrated example generally includes a handle 204, a first elongated shaft 206 (which comprises an outer shaft in the illustrated example) extending distally from the handle 204, at least one actuator assembly 208 extending distally through the first shaft 206, a second elongated shaft 209 (which comprises an inner shaft in the illustrated example) extending through the first shaft 206, and a nosecone 210 coupled to a distal end portion of the second shaft 209. The second shaft 209 and the nosecone 210 can define a guidewire lumen for advancing the delivery apparatus through a patient's vasculature over a guidewire. The at least one actuator assembly 208 can be configured to radially expand and/or radially collapse the prosthetic valve 202 when actuated, such as by one or more knobs 211, 212, 214 included on the handle 204 of the delivery apparatus 200.

Though the illustrated example shows two actuator assemblies 208 for purposes of illustration, it should be understood that one actuator assembly 208 can be provided for each actuator (e.g., actuator or threaded rod 126) on the prosthetic valve. For example, three actuator assemblies 208 can be provided for a prosthetic valve having three actuators. In some examples, a greater or fewer number of actuator assemblies can be present.

In some examples, a distal end portion 216 of the shaft 206 can be sized to house the prosthetic valve in its radially compressed, delivery state during delivery of the prosthetic valve through the patient's vasculature. In this manner, the distal end portion 216 functions as a delivery sheath or capsule for the prosthetic valve during delivery,

The actuator assemblies 208 can be releasably coupled to the prosthetic valve 202. For example, in the illustrated example, each actuator assembly 208 can be coupled to a respective actuator (e.g., threaded rod 126) of the prosthetic valve 202. Each actuator assembly 208 can comprise a support tube and an actuator member. When actuated, the actuator assembly can transmit pushing and/or pulling forces to portions of the prosthetic valve to radially expand and collapse the prosthetic valve as previously described. The actuator assemblies 208 can be at least partially disposed radially within, and extend axially through, one or more lumens of the first shaft 206. For example, the actuator assemblies 208 can extend through a central lumen of the shaft 206 or through separate respective lumens formed in the shaft 206.

The handle 204 of the delivery apparatus 200 can include one or more control mechanisms (e.g., knobs or other actuating mechanisms) for controlling different components of the delivery apparatus 200 in order to expand and/or deploy the prosthetic valve 202. For example, in the illustrated example the handle 204 comprises first, second, and third knobs 211, 212, and 214, respectively.

The first knob 211 can be a rotatable knob configured to produce axial movement of the first shaft 206 relative to the prosthetic valve 202 in the distal and/or proximal directions in order to deploy the prosthetic valve from the delivery sheath 216 once the prosthetic valve has been advanced to a location at or adjacent the desired implantation location with the patient's body. For example, rotation of the first knob 211 in a first direction (e.g., clockwise) can retract the sheath 216 proximally relative to the prosthetic valve 202 and rotation of the first knob 211 in a second direction (e.g., counter-clockwise) can advance the sheath 216 distally. In some examples, the first knob 211 can be actuated by sliding or moving the first knob 211 axially, such as pulling and/or pushing the knob. In some examples, actuation of the first knob 211 (rotation or sliding movement of the first knob 211) can produce axial movement of the actuator assemblies 208 (and therefore the prosthetic valve 202) relative to the delivery sheath 216 to advance the prosthetic valve distally from the sheath 216.

The second knob 212 can be a rotatable knob configured to produce radial expansion and/or compression of the prosthetic valve 202. For example, rotation of the second knob 212 can rotate the threaded rods of the prosthetic valve 202 via the actuator assemblies 208. Rotation of the second knob 212 in a first direction (e.g., clockwise) can radially expand the prosthetic valve 202 and rotation of the second knob 212 in a second direction (e.g., counter-clockwise) can radially collapse the prosthetic valve 202.

In some examples, the second knob 212 can be actuated by sliding or moving the second knob 212 axially, such as pulling and/or pushing the knob.

The third knob 214 can be a rotatable knob operatively connected to a proximal end portion of each actuator assembly 208. The third knob 214 can be configured to retract an outer sleeve or support tube of each actuator assembly 208 to disconnect the actuator assemblies 208 from the proximal portions of the actuators of the prosthetic valve (e.g., threaded rod). Once the actuator assemblies 208 are uncoupled from the prosthetic valve 202, the delivery apparatus 200 can be removed from the patient, leaving just the prosthetic valve 202 in the patient.

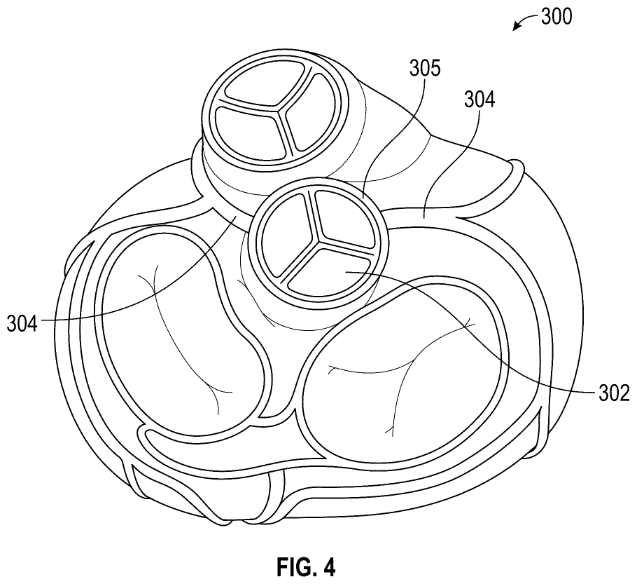

In some examples, the delivery apparatus 200 (or another, similar delivery apparatus) can be configured to deploy and implant a prosthetic heart valve (e.g., prosthetic valve 100 of FIGS. 1A and 1B) in the native aortic annulus of a native aortic valve. An exemplary heart 300 including an aortic valve 302 is shown in FIG. 4. As shown in FIG. 4, two coronary arteries (e.g., the left coronary artery and the right coronary artery) 304 are coupled to and branch off from the aorta 305, proximate to the aortic valve 302. The coronary arteries 304 carry oxygenated blood from the aorta to the muscle of the heart 300.

FIGS. 5A and 5B are cross-sectional views of an exemplary aorta 305 and aortic valve illustrating different circumferential orientations of a prosthetic heart valve 306 implanted within the aortic valve (relative to native commissures 312 of the aortic valve, as described further below). In a first example shown in FIG. 5A, if the prosthetic heart valve 306 is implanted within the aortic valve with one or more commissures 310 of the prosthetic heart valve 306 being circumferentially aligned with (and thus potentially arranged in front of and at least partially blocking) the coronary arteries 304, blood flow to the coronary arteries 304 can be reduced. For example, since adjacent leaflets of the prosthetic heart valve 306 are coupled together at the commissures 310, the commissures 310 can partially block and/or reduce blood flow through the portions of the frame of the prosthetic heart valve 306 to which they are coupled. Thus, less oxygenated blood flow can reach the coronary arteries 304 and the heart muscle.

Thus, instead of deploying the prosthetic heart valve with the delivery apparatus in a random rotational orientation relative to the aorta 305, which may result in commissures 310 of the prosthetic heart valve 306 being arranged in front of the coronary arteries 304 (as shown in FIG. 5A), it may be desirable to deploy the prosthetic heart valve 306 in an targeted rotational orientation where the commissures 310 are positioned away from and do not inhibit flow to the coronary arteries 304 (as shown in FIG. 5B). For example, as shown in FIG. 5B, if the prosthetic heart valve 306 were implanted in the aortic valve such that commissures 310 of the radially expanded prosthetic heart valve 306 were circumferentially aligned with the native commissures 312 of the aortic valve, then blood flow to the coronary arteries 304 may not be reduced.

Further, in some instances, a second prosthetic heart valve may be implanted within a previously implanted first prosthetic heart valve (which can be referred to as a “valve-in-valve” procedure). During such valve-in-valve procedures it may be desirable to implant the second prosthetic heart valve such that its commissures align with the commissures of the first prosthetic heart valve.

Thus, it is desirable to easily visualize the commissures of a prosthetic heart valve such that the prosthetic heart valve can be implanted with a specified rotational orientation relative to the anatomy (e.g., such that commissures of the prosthetic heat valve are in alignment with commissures of the native valve or a previously implanted valve).

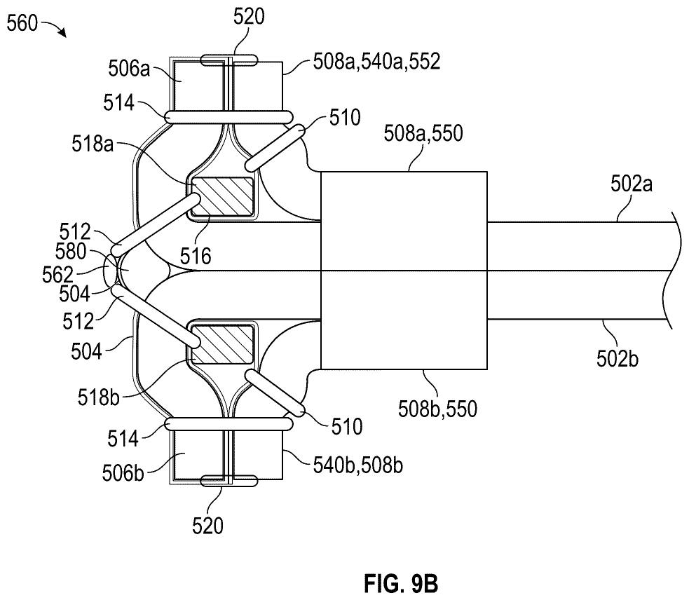

In some examples, a radiopaque marker can be positioned on a prosthetic valve, such as on or near a commissure (or at each commissure) of the prosthetic valve, as shown in FIGS. 6 and 9A-9C). As a result, a location of a selected commissure (or two or more commissures) of the prosthetic valve, to which the radiopaque marker(s) is coupled, can be visualized by medical imaging (e.g., fluoroscopy) both prior to implantation in a heart (e.g., when the prosthetic valve is in a radially compressed state) and following implantation in the heart (e.g., when the prosthetic valve is in a radially expanded state).

In some examples, the radiopaque marker(s) at the commissure(s) of the prosthetic valve can be visualized under fluoroscopy both before and after radial expansion of the prosthetic valve (during and after implantation with a delivery apparatus). For example, one or more radiopaque markers positioned at one or more commissures of the prosthetic valve can be visualized after implantation when the prosthetic valve is in a radially expanded state (e.g., during future interventions to locate the commissures of the implanted prosthetic valve and/or to confirm the location of the commissures of the implanted prosthetic valve relative to the native valve commissures). In some examples, one or more radiopaque markers positioned at one or more commissures of the prosthetic valve can be visualized under fluoroscopy while the prosthetic valve is radially compressed on a delivery apparatus (e.g., delivery apparatus 200 of FIG. 3) while inside of a patient, even when a delivery sheath or capsule of the delivery apparatus (e.g., the distal end portion 216 of the shaft 206 of the delivery apparatus 200) is covering the radially compressed prosthetic valve.

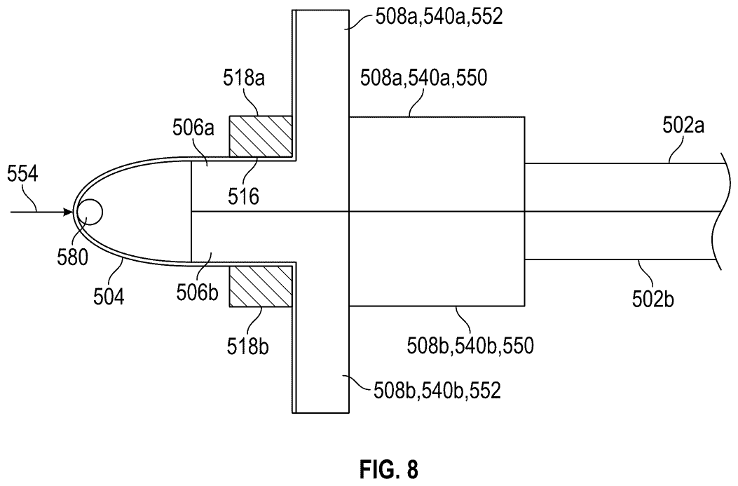

FIG. 6 shows an exemplary radiopaque maker 400 attached to a commissure 152 of the prosthetic valve 100. As introduced above, a commissure 152 can comprise a first commissure tab 402 of a first leaflet 158 paired with a second commissure tab 404 of a second leaflet 158, the first and second leaflets 158 disposed adjacent to one another inside the frame 102. During assembly of the prosthetic valve, the pair of commissure tabs 402 and 404 of the two adjacent leaflets 158 can then be inserted through a commissure opening 146 (or commissure window) defined by a commissure support structure 144 of the frame 102. As a result, the first and second commissure tabs 402 and 404 extend through the commissure opening 146 and are arranged exterior to the frame 102 (extending radially outward from an outer surface of the frame 102). The first and second commissure tabs 402 and 404 forming the commissure 152 can then be secured to the commissure support structure 144, on an outside of the frame 102. In some examples, one or more pieces of fabric or wedge members can be used to cover and/or secure a portion of the commissure 152 to the commissure support structure 144, as described further below with reference to FIGS. 7-9C.

As shown in FIG. 6, the radiopaque marker 400 is attached to an outer surface (a radially outward facing surface) of the commissure 152 such that the radiopaque marker 400 is exposed. In some examples, a piece of fabric or other flexible member can be secured to the commissure 152 and cover all or a portion of the radiopaque marker.

In the example of FIG. 6, the radiopaque marker 400 is attached to the commissure 152 with one or more sutures 406 that extend through an aperture 410 (e.g., a central aperture) in the radiopaque marker 400. In the illustrated example, the radiopaque marker 400 is ring-shaped and has a central aperture 410.

However, in some examples, the radiopaque marker 400 can have different shapes, such as square, oval, arc-shaped, or the like, having an aperture for receiving a suture.

In the example shown in FIG. 6, the radiopaque marker 400 has a smaller diameter or length (in an axial direction) than an axial length 408 of the commissure 152 and commissure tabs 402 and 404. In some examples, an axial length or diameter of the radiopaque marker 400 can be larger than shown in FIG. 6, and in some examples, can be the same length as the axial length 408 of the commissure 152.

While only one radiopaque marker 400 is visible in FIG. 6, it should be noted that one or more of the commissures 152 can include a radiopaque marker attached thereto. For example, in some cases, a radiopaque marker can be secured to each commissure 152 of the prosthetic valve 100 (e.g., the prosthetic valve 100 can include three radiopaque markers: one for each commissure).

In some examples, a radiopaque marker may only be attached to one or two of the commissures 152.

In some examples, the radiopaque marker 400 and the other radiopaque markers described herein can comprise a radiopaque material, such as tantalum. In some examples, the radiopaque markers described herein can comprise another type of radiopaque material or combination of materials, such as one or more of iodine, barium, barium sulfate, tantalum, bismuth, or gold.

In this way, the radiopaque marker(s) can be visualized under medical imaging (e.g., fluoroscopy) when the prosthetic valve is in a radially compressed state on a delivery apparatus and/or when the prosthetic valve is in a radially expanded state after being implanted with the delivery apparatus. The one or more radiopaque markers allow for a location of one or more of the commissures of the prosthetic valve to be visualized and located during and after an implantation procedure. As such, a position of the one or more commissures of the prosthetic valve can be determined and/or manipulated (while still on a delivery apparatus) relative to the native anatomy and/or a previously implanted prosthetic valve.

Radiopaque markers for a commissure of a prosthetic valve can be attached to the commissure in different ways and in different locations relative to the components of the commissure. FIGS. 7, 8, and 9A-9C show an exemplary method of forming a commissure 560 and securing the commissure 560 to a frame of a prosthetic valve. FIGS. 9A-9C also show three different exemplary positions for the marker at or within the commissure.

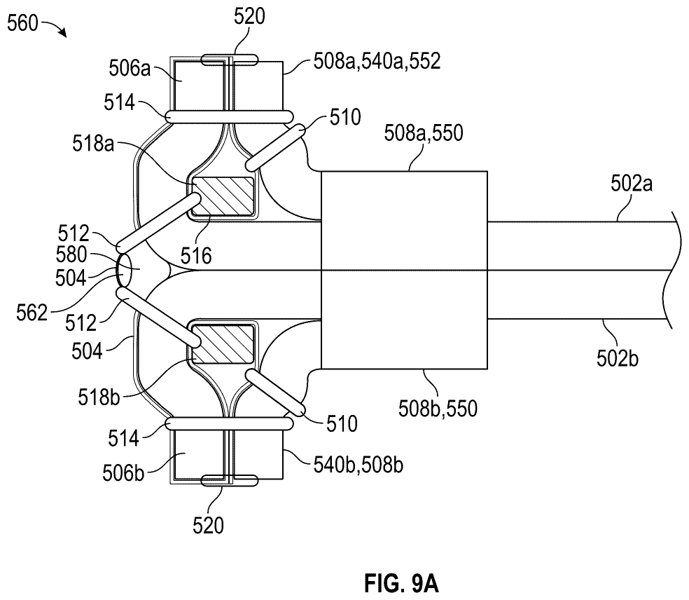

Turning first to FIG. 7, two leaflets 502a, 502b of a leaflet assembly for a prosthetic heart valve (such as prosthetic valve 100) are shown, each leaflet 502a, 502b, including primary tabs 506a, 506b (which are the lower tabs before folding) at opposite sides of the leaflet and secondary tabs 508a, 508b (which are the upper tabs before folding) at an outflow end of the leaflet where a leaflet free edge is located. In particular examples, a leaflet assembly or valvular structure for a prosthetic heart valve can be formed by connecting a flexible connector 504 (also referred to herein as a “flexible member”) to the pair of leaflets 502a, 502b at the primary tab 506a of the leaflet 502a and the primary tab 506b of the leaflet 502b. The flexible connector 504 can be connected to the primary tabs 506a, 506b with sutures. The flexible connector 504 can comprise, for example, a piece of fabric (e.g., PET fabric). In some examples, a wedge element (or member) 580 (FIG. 8) can be connected to one side of the flexible connector 504. The wedge element 580 can comprise, for example, a relatively heavy gauge suture, such as a braided suture (e.g., an Ethibond suture), or a piece of fabric.

A third leaflet (not shown in FIGS. 7 and 8) can be similarly coupled to leaflets 102a, 102b by connecting a second connector 504 to the primary tab 506b of the leaflet 502a and to a primary tab of the third leaflet and connecting a third connector 504 to the primary tab 506a of the leaflet 502b and to the other primary tab of the third leaflet, thereby forming a leaflet assembly of three leaflets (similar to the assembly shown in FIGS. 1A and 1B) coupled to each other with respective connectors 504.

The secondary tabs 508a, 508b of each leaflet can then be folded downwardly (in the view of FIG. 7) against their corresponding primary tabs 506a, 506b. For example, referring to FIG. 7, the secondary tab 508a of the leaflet 502a can be folded downwardly against the primary tab 506a of the leaflet 502a on the same side of the leaflet as the connector 504. In this manner, a second tab portion 540a of the secondary tab 508a can partially overlap a portion of the connector 504 (i.e., a portion of the connector 504 is situated between the primary tab 506a and the second tab portion 540a). Similarly, the secondary tab 508b of the leaflet 502b can be folded downwardly against the primary tab 506b of the leaflet 502b.

In some examples, the primary tabs 506a, 506b, or the primary tabs 506a, 506b together with the secondary tabs 508a, 508b, can be referred to herein as commissure tabs (such as commissure tabs 402 and 406 in FIG. 6).

After folding the secondary tabs 508a, 508b, each of the second tab portions 540a, 540b can be folded lengthwise along a vertical fold axis to form an L-shape having an inner portion 550 and an outer portion 552 (FIG. 8). The inner portion 550 can contact the inner surface of the leaflet and the outer portion 552 can contact the connector 504. The outer portions 552 can be sutured to the connector 504, such as with sutures 510 (shown in FIGS. 9A-9C)

Referring now to FIG. 8, a commissure tab assembly formed with the connector 504, the tabs 506a, 508a of the leaflet 502a, and the tabs 506b, 508b of the leaflet 502b can be coupled to a commissure window 516 of a frame as follows. As shown in FIG. 8, the connector 504 and the primary tabs 506a, 506b can be inserted through the commissure window 616 defined by a pair of strut portions 518a, 518b (from the inside of the frame to the outside of the frame), while the secondary tabs 508a, 508b remain inside the frame. The strut portions 518a and 518b can be part of a commissure support structure of the frame, such as commissure support structure 144 shown in FIGS. 1A, 2A, 2B, and 6.

The commissure tab assembly is then pressed inwardly at the wedge element 580 (in the direction of arrow 554) such that the outer portion 552 of the secondary tab portion 540a and a portion of the connector 504 are disposed against the frame (an inner surface of the strut portion 518a) on one side of the commissure window 516 and the outer portion 552 of the secondary tab portion 540b and a portion of the connector 504 are disposed against the frame (an inner surface of the strut portion 518b) on the other side of the commissure window 516.

As shown in FIGS. 9A-9C, the pressing of the commissure tab assembly also causes the primary tab 506a and a portion of the connector 504 to fold around the strut portion 518a on the outside of the frame opposite the outer portion 552 of the secondary tab 508a, and the primary tab 506b and a portion of the connector 504 to fold around the strut portion 518b on the outside of the frame opposite the outer portion 552 of the secondary tab 508b. Further, as shown in FIGS. 9A and 9B, the wedge element 580 is disposed against outer surfaces of the primary tabs 506a, 506b, exterior to the commissure window 516 and at a location where the primary tabs 506a, 506b separate from one another to extend around respective outer surfaces of the strut portions 518a, 518b of the commissure support.

A pair of suture lines 512 can be formed to retain the primary tabs 506a, 506b against the frame. Each suture line 512 can extend through the connector 504, a primary tab, the wedge element 580 (FIGS. 9A and 9B), and another portion of the connector 504.

Each primary tab 506a, 506b can be secured to a corresponding secondary tab 508a, 508b (on an outside edge of the strut portions 518a and 518b) with a primary suture line 514. Each primary suture line 514 extends through a first layer of the connector 504, a corresponding primary tab 506a, 506b, a second layer of the connector 504, a third layer of the connector 504, and the outer portion 552 of the corresponding secondary tab 508a, 508b. The end portions of the suture material used to form the primary suture lines 514 (or separate sutures) can be used to form whip stitches 520 at the adjacent outer edges of the tabs 506a, 508a and at the adjacent outer edges of the tabs 506b, 508b. A first set of stitches 520 can extend through the tabs 506a, 508a and two layers of the connector 504 between the tabs 506a, 508a, and a second set of stitches 520 can extend through the tabs 506b, 508b and two layers of the connector 504 between the tabs 506b, 508b.

The remaining commissure tab assemblies of the leaflet assembly can be coupled to respective commissure windows of the frame (such as the commissure windows 146 of the frame 102) in the same manner as described above. Further details of the method for forming the commissure tab assemblies and coupling them to the frame are disclosed in U.S. Pat. No. 9,393,110, which is incorporated herein by reference. It should be noted that FIGS. 7-9C show one exemplary technique for coupling the commissures of a leaflet assembly to a frame. Other techniques, methods, and mechanisms can be used for coupling the commissures of the leaflet assembly to the frame, such as any of those disclosed in U.S. Pat. No. 9,393,110, U.S. Publication No. 2018/0325665, or U.S. Application No. 63/003,085, filed Mar. 31, 2020, which are incorporated herein by reference.

Turning again to FIGS. 9A-9C, three different examples of a location or positioning of a radiopaque marker 562 at the commissure 560 is shown. The marker 562 can comprise any of the radiopaque materials described herein, such as tantalum.

In one example, as shown in FIG. 9A, the marker 562 can be positioned between the wedge element 580 and an inner surface of the flexible connector 504. In such examples, one or more stitches of one or both of the suture lines 512 can also pass through or around a portion of the marker 562 (such as through an aperture in the marker 562).

In some examples, one or more additional sutures can be used to secure the marker 562 to the wedge element 580 and/or the flexible connector 504.

In some examples, as shown in FIG. 9B, the marker 562 can be positioned on an exterior or outer surface of the flexible connector 504. In some examples, the marker 562 can be positioned against the outer surface of the flexible connector 504 and attached to the flexible connector 504 via one or more fasteners (e.g., sutures). For example, the marker 562 can be secured to the flexible connector 504 in a similar manner to that shown in FIG. 6 where sutures 406 secure the marker 400 to the commissure 152. As a result, the marker 562 can be coupled to an outside of the commissure 560. In some examples, one or more stitches of one or both of the suture lines 512 can also pass through or around a portion of the marker 562, after passing through the flexible connector 504.

In some examples, as shown in FIG. 9C, the marker 562 can replace the wedge element 580. Thus, the marker 562 can be sized and shaped such that it functions as both a radiopaque marker and a wedge element that is positioned against the primary tabs 506a, 506b and underneath (or interior to) the flexible connector 504. In some examples, one or more stitches of one or both of the suture lines 512 can also pass through or around a portion of the marker 562 (such as through an aperture in the marker 562).

In some examples, one or more additional sutures can be used to secure the marker 562 to the flexible connector 504 and/or the primary tabs 506a, 506b.

Though the marker 562 is shown with an oval-shaped cross-section in FIGS. 9A-9C, it should be noted that the marker 562 can have various shapes as long as it's shape and/or size allows it to be connected to the commissure 560, as described above. For example, in some prosthetic valves, the marker 562 can be smaller or larger than shown in FIGS. 9A-9C. Further, in some examples, an axial length of the marker 562 can vary. For example, the marker 562 can extend along an entire axial length of the commissure 560 or the marker 562 can only extend along a portion of the axial length of the commissure 560 (such as shown in FIG. 6 for marker 400 and commissure 152).

In some examples, for all of the different exemplary positions for the marker at or within the commissure described herein (e.g., with reference to FIGS. 6 and 9A-9C), the marker may not be in direct contact with any portion of the frame.

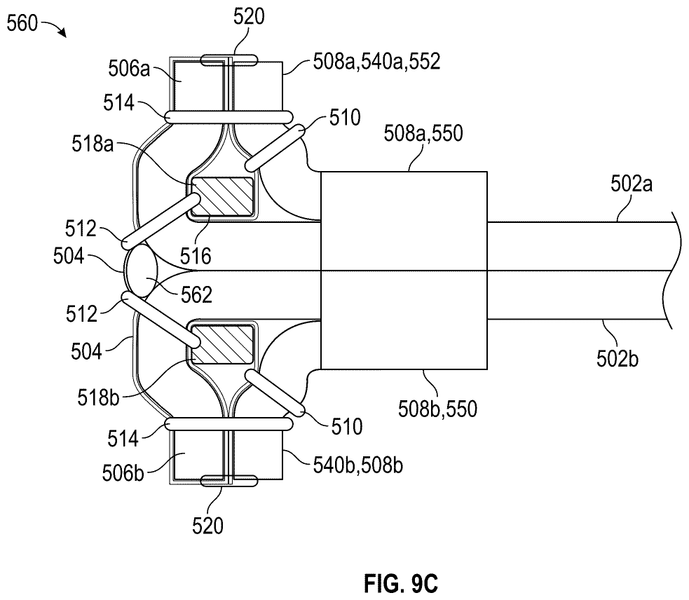

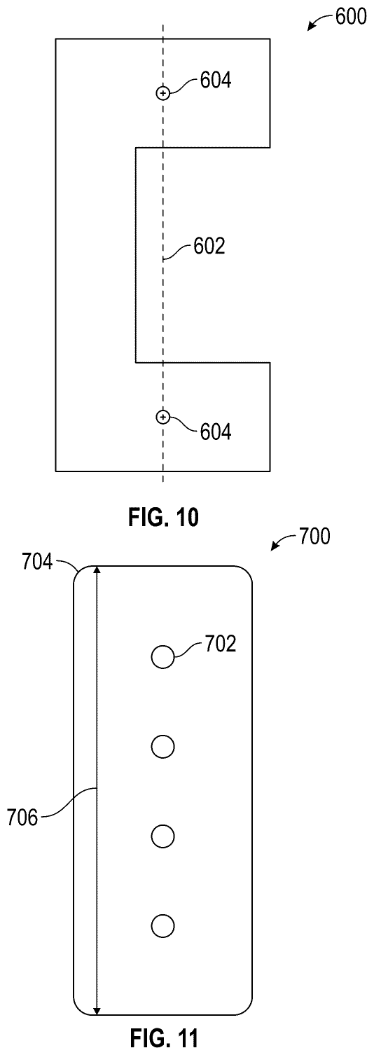

FIGS. 10 and 11 show different examples of a radiopaque marker configured to be coupled to a commissure of a prosthetic valve. In some examples, the markers shown in FIGS. 10 and 11 can be used in place of the marker 562 in FIGS. 9A-9C or marker 400 in FIG. 6.

FIG. 10 shows an example of a radiopaque marker 600 that is configured to be coupled to a commissure of a prosthetic valve. The marker 600 can be attached to a commissure in various locations at or within the commissure, such as one of the locations described above with reference to FIGS. 6 and 9A-9C. Further, the marker 600 can comprise any of the radiopaque materials described herein, such as tantalum.

As shown in FIG. 10, the marker 600 can be C-shaped (shaped as a letter “C”). Such a C-shape can be reflection asymmetric across a central longitudinal axis 602 of the marker 600.

Thus, the marker 600 can be an asymmetric marker. Such asymmetry can allow a position of the marker (and thus the commissure of the valve to which it is attached) to be distinguishable within an imaging view. For example, the C-shaped asymmetric marker 600 can be in a first orientation which is its forward-readable orientation (e.g., appears in its correct, not backward, orientation to a reader as the letter “C”). If the C-shaped asymmetric marker 600 were rotated by approximately 180 degrees around its longitudinal axis 602, the C-shaped asymmetric marker 600 would be in a second orientation and the “C” would appear backward (e.g., flipped). These two orientations of the C-shaped asymmetric marker 600 can be seen in a medical imaging view (e.g., using fluoroscopy). The two orientations of the C-shaped asymmetric marker (and other asymmetric markers described herein) can be mirror images of one another. As such, it can be determined whether the marker 600 is in a front or back of an imaging view, and thus, a position of a commissure to which the marker 600 is attached can be better determined relative to the native anatomy.

In some examples, the marker 600 can have a different shape, such as being shaped as a different letter of the alphabet (e.g., shaped as an “e”, “E”, “P”, “L), or having an oval, circular, ring, square, or arc shape.

In some examples, the marker 600 can include one or more apertures 604 that are configured to receive a fastener (such as a suture) for attaching the marker 600 to a commissure of a prosthetic heart valve (such as one of the sutures shown in FIGS. 6 and 9A-9C). While FIG. 10 shows two apertures 604 in the marker 600, in some examples, the marker 600 can include more or less than two apertures 604 (e.g., one more central aperture or three, four, or the like, apertures). Further, a diameter of the one or more aperture 604 can vary based on a size and/or number of sutures to be threaded through the aperture for attaching the marker 600 to a commissure (e.g., the diameter of apertures 604 can be smaller or larger than shown in FIG. 10).

FIG. 11 shows an example of a radiopaque marker 700 that is configured to be coupled to a commissure of a prosthetic valve. The marker 700 can be attached to a commissure in various locations at or within the commissure, such as one of the locations described above with reference to FIGS. 6 and 9A-9C. Further, the marker 700 can comprise any of the radiopaque materials described herein, such as tantalum.

As shown in FIG. 11, the marker 700 is rectangular and comprises one or more apertures 702 that are configured to receive a fastener (such as a suture) for attaching the marker 700 to a commissure of a prosthetic heart valve (such as one of the sutures shown in FIGS. 6 and 9A-9C). While FIG. 11 shows four apertures 702 in the marker 700, in some examples, the marker 700 can include more or less than four apertures 702 (e.g., one, two, three, five, or the like). Further, a diameter of the one or more aperture 702 can vary based on a size and/or number of sutures to be threaded through the aperture for attaching the marker 700 to a commissure (e.g., the diameter of apertures 702 can be smaller or larger than shown in FIG. 11).

In some examples, as shown in FIG. 11, edges 704 of the marker 700 can be rounded or filleted, thereby providing smoother edges for interfacing with a commissure of the prosthetic heart valve when attached thereto. Further, a length 706 (in an axial direction) of the marker 700 can be selected based on an axial length of the commissure to which it is to be attached. For example, the length 706 can be selected such that it is the same or shorter than an axial length of the commissure (or the commissure tabs of the commissure, such as the axial length 408 of the commissure 152 in FIG. 6).

Additional examples of radiopaque markers for prosthetic heart valves and implanting prosthetic heart valves using radiopaque markers to align commissures of the prosthetic heart valve relative to selected anatomical features can be found in International Patent Application No. PCT/US2021/047063, filed Aug. 23, 2021, which is incorporated herein by reference.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (e.g., by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic heart valve comprising: an annular frame comprising a plurality of struts; a plurality of leaflets arranged within the frame; at least one commissure comprising commissure tabs of two adjacent leaflets of the plurality of leaflets connected to each other, the at least one commissure coupled to the frame; and at least one radiopaque marker, separate from the frame, attached to the commissure, wherein the marker is configured to indicate a location of the commissure of the prosthetic heart valve.

Example 2. The prosthetic heart valve of any example herein, particularly example 1, wherein the marker is sewn to an exterior surface of the commissure that faces radially outward from the frame.

Example 3. The prosthetic heart valve of any example herein, particularly either example 1 or example 2, wherein the marker is sewn to a radially outward facing surface of a flexible member that is connected to the commissure tabs and forms a part of the commissure.

Example 4. The prosthetic heart valve of any example herein, particularly example 3, wherein the flexible member is a piece of fabric.

Example 5. The prosthetic heart valve of any example herein, particularly example 1, wherein the frame comprises a plurality of commissure supports, wherein each corresponding commissure support defines a commissure window in the frame, wherein at least a portion of the commissure tabs extend radially through the commissure window and are secured to and around outer surfaces of strut portions of the corresponding commissure support which define the commissure window, and wherein the commissure includes a flexible member that covers outer surfaces of the commissure tabs on an exterior of the frame.

Example 6. The prosthetic heart valve of any example herein, particularly example 5, wherein the marker serves as a wedge element that is disposed against the outer surfaces of the commissure tabs, exterior to the commissure window and at a location where the commissure tabs separate from one another to extend around respective outer surfaces of the strut portions of the corresponding commissure support, and wherein the marker is disposed between the flexible member and the commissure tabs.

Example 7. The prosthetic heart valve of any example herein, particularly example 5, further comprising a wedge element secured to the commissure tabs exterior to the commissure window and at a location where the commissure tabs separate from one another to extend around respective outer surfaces of the strut portions of the corresponding commissure support, and wherein the wedge element is disposed between the commissure tabs and the flexible member.

Example 8. The prosthetic heart valve of any example herein, particularly example 7, wherein the marker is disposed between the wedge element and the flexible member.

Example 9. The prosthetic heart valve of any example herein, particularly example 7, wherein the marker is attached to an outer surface of the flexible member, the flexible member disposed between the marker and the wedge element.

Example 10. The prosthetic heart valve of any example herein, particularly any one of examples 5-9, wherein the marker is secured to the flexible member and the commissure tabs via one or more sutures.

Example 11. The prosthetic heart valve of any example herein, particularly any one of examples 1-10, wherein the marker is reflection asymmetric across an axis that is parallel to a central longitudinal axis of the frame, the central longitudinal axis extending between an inflow end and an outflow end of the frame.

Example 12. The prosthetic heart valve of any example herein, particularly any one of examples 1-11, wherein the marker is C-shaped.

Example 13. The prosthetic heart valve of any example herein, particularly any one of examples 1-12, wherein the marker is rectangular with rounded edges.

Example 14. The prosthetic heart valve of any example herein, particularly any one of examples 1-13, wherein the marker comprises tantalum.

Example 15. The prosthetic heart valve of any example herein, particularly any one of examples 1-14, wherein the marker comprises one or more apertures configured to receive a suture for fastening the marker to the commissure.

Example 16. The prosthetic heart valve of any example herein, particularly any one of examples 1-15, wherein the frame is radially compressible and expandable between a radially compressed configuration and a radially expanded configuration.

Example 17. The prosthetic heart valve of any example herein, particularly any one of examples 1-16, wherein the frame comprises one or more mechanical expansion and locking mechanisms configured to radially expand and compress the frame.

Example 18. The prosthetic heart valve of any example herein, particularly any one of examples 1-17, wherein the marker is not in direct contact with any portion of the frame.

Example 19. A prosthetic heart valve comprising: a frame comprising an inflow end, an outflow end, and a plurality of support posts including a commissure window; a plurality of leaflets arranged within the frame and configured to regulate a flow of blood through the frame in one direction between the inflow end and outflow end; at least one commissure comprising commissure tabs of two adjacent leaflets of the plurality of leaflets connected to each other and extending radially through the commissure window of a corresponding support post of the plurality of support posts, the at least one commissure connected to the corresponding support post; and a radiopaque marker attached to the commissure, wherein the marker is configured to indicate a location of the commissure of the prosthetic heart valve.

Example 20. The prosthetic heart valve of any example herein, particularly example 19, wherein the marker is attached to the commissure exterior to the frame and on a radially outward facing surface of the commissure.

Example 21. The prosthetic heart valve of any example herein, particularly example 19, wherein the commissure comprises a wedge element disposed against the commissure tabs on an exterior of the commissure window and a flexible member covering the commissure tabs on the exterior of the commissure window, the wedge element disposed interior to the flexible member relative to a central longitudinal axis of the frame.

Example 22. The prosthetic heart valve of any example herein, particularly example 21, wherein the marker is attached to an outer surface of the flexible member.

Example 23. The prosthetic heart valve of any example herein, particularly example 21, wherein the marker is disposed between the flexible member and the wedge element.

Example 24. The prosthetic heart valve of any example herein, particularly example 21, wherein the marker is the wedge element and is disposed between the commissure tabs and the flexible member.

Example 25. The prosthetic heart valve of any example herein, particularly any one of examples 21-24, wherein the flexible member comprises a piece of fabric.

Example 26. The prosthetic heart valve of any example herein, particularly any one of examples 19-25, wherein the marker is reflection asymmetric across an axis that is parallel to a central longitudinal axis of the frame, the central longitudinal axis extending between the inflow end and an outflow end of the frame.

Example 27. The prosthetic heart valve of any example herein, particularly any one of examples 19-26, wherein the marker is shaped as a letter of the alphabet.

Example 28. The prosthetic heart valve of any example herein, particularly example 27, wherein the marker is C-shaped.

Example 29. The prosthetic heart valve of any example herein, particularly any one of examples 19-25, wherein the marker is rectangular with rounded edges.

Example 30. The prosthetic heart valve of any example herein, particularly any one of examples 19-29, wherein the marker comprises a plurality of apertures spaced apart along an axial length of the marker.

Example 31. The prosthetic heart valve of any example herein, particularly any one of examples 19-25, wherein the marker is ring-shaped with a central aperture and wherein the marker is attached to the commissure via one or more sutures extending through the central aperture.

Example 32. The prosthetic heart valve of any example herein, particularly any one of examples 19-31, wherein the marker comprises tantalum.

Example 33. The prosthetic heart valve of any example herein, particularly any one of examples 19-32, wherein the marker comprises one or more apertures configured to receive a suture for fastening the marker to the commissure.

Example 34. The prosthetic heart valve of any example herein, particularly any one of examples 19-33, wherein the frame comprises one or more mechanical expansion and locking mechanisms configured to radially expand and compress the frame.

Example 35. The prosthetic heart valve of any example herein, particularly example 34, wherein the one or more mechanical expansion and locking mechanisms comprise a plurality of mechanical expansion and locking mechanisms that are arranged in alternating order with the plurality of support posts around a circumference of the frame.

Example 36. The prosthetic heart valve of any example herein, particularly any one of examples 19-35, further comprising a skirt arranged around an outer surface of the frame at the inflow end of the frame.

Example 37. A prosthetic heart valve comprising: a frame comprising: a plurality of support posts spaced apart about a circumference of the frame, each support post of the plurality of support posts including a commissure window; and one or more expansion and locking mechanisms that are configured to radially expand and/or compress the frame, each of the one or more expansion and locking mechanism comprising a pair of axially aligned posts and a threaded rod extending through the pair of posts; a plurality of leaflets arranged within the frame and configured to regulate a flow of blood through the frame in one direction; at least one commissure comprising commissure tabs of two adjacent leaflets of the plurality of leaflets connected to each other and extending radially through the commissure window of a corresponding support post, the at least one commissure connected to the corresponding support post; and a radiopaque marker attached to the commissure.

Example 38. The prosthetic heart valve of any example herein, particularly example 37, wherein the commissure comprises a wedge element disposed against and between the commissure tabs on an exterior of the commissure window and a flexible member covering at least a portion of the commissure tabs on the exterior of the commissure window, the wedge element disposed between the flexible member and the commissure tabs.

Example 39. The prosthetic heart valve of any example herein, particularly example 38, wherein the marker is attached to an outer surface of the flexible member that faces away from the frame.

Example 40. The prosthetic heart valve of any example herein, particularly example 38, wherein the marker is disposed between the flexible member and the wedge element.

Example 41. The prosthetic heart valve of any example herein, particularly example 37, wherein the marker is configured as a wedge element that is disposed against and between the commissure tabs on an exterior of the commissure window, and wherein the commissure comprises a flexible member covering at least a portion of the commissure tabs on the exterior of the commissure window, the marker disposed between the flexible member and the commissure tabs.

Example 42. The prosthetic heart valve of any example herein, particularly any one of examples 37-41, wherein the marker comprises tantalum.

Example 43. The prosthetic heart valve of any example herein, particularly any one of examples 37-42, wherein the marker comprises one or more apertures configured to receive a suture for fastening the marker to the commissure.

Example 44. The prosthetic heart valve of any example herein, particularly example 43, wherein the marker is rectangular with rounded edges and comprises a plurality of apertures spaced apart along its axial length.

Example 45. The prosthetic heart valve of any example herein, particularly any one of examples 37-43, wherein the marker is reflection asymmetric across an axis that is parallel to a central longitudinal axis of the frame, the central longitudinal axis extending between an inflow end and an outflow end of the frame.

Example 46. The prosthetic heart valve of any example herein, particularly example 45, wherein the marker is shaped as a letter of the alphabet.

Example 47. The prosthetic heart valve of any example herein, particularly example 46, wherein the marker is C-shaped.

Example 48. The prosthetic heart valve of any example herein, particularly any one of examples 37-47, wherein the pair of axially aligned posts extend between an inflow end and an outflow end of the frame and are axially movable relative to one another to permit the frame to radially expand and/or compress.

Example 49. The prosthetic heart valve of any example herein, particularly example 48, wherein the pair of axially aligned posts includes a first post comprising an inner bore and a second post, wherein the threaded rod extends through the inner bore of the first post and is coupled to the second post, and wherein the threaded rod is configured to radially expand and/or radially compress the frame only when rotated but is otherwise configured to prevent radial expansion and/or compression of the prosthetic heart valve.

Example 50. The prosthetic heart valve of any example herein, particularly either example 48 or example 49, wherein the frame comprises a plurality of expansion and locking mechanisms that are spaced about the circumference of the frame.

Example 51. The prosthetic heart valve of any example herein, particularly example 50, wherein each support post is disposed between two expansion and locking mechanisms of the plurality of expansion and locking mechanisms.

Example 52. A method comprising: at a native valve of a heart, visualizing under fluoroscopy a position of a radiopaque marker attached to a commissure of a prosthetic heart valve relative to the native valve, wherein the commissure comprises commissure tabs of two adjacent leaflets of a plurality of leaflets of the prosthetic heart valve connected to each other, the commissure connected to a frame of the prosthetic heart valve, and wherein the marker is separate from the frame; and implanting the prosthetic heart valve into the native valve or a previously implanted prosthetic heart valve such that the commissure of the prosthetic heart valve to which the marker is attached is aligned with the native commissure of the native valve or a selected commissure of the previously implanted prosthetic heart valve.

Example 53. The method of any example herein, particularly example 52, wherein the implanting includes radially expanding the prosthetic heart valve from a radially compressed state to a radially expanded state with a delivery apparatus.

Example 54. The method of any example herein, particularly example 53, wherein radially expanding the prosthetic heart valve includes using an actuator assembly of the delivery apparatus to actuate an expansion and locking mechanism integral with the frame of the prosthetic heart valve to radially expand the prosthetic heart valve.

Example 55. The method of any example herein, particularly any one of examples 52-53, further comprising, after implanting the prosthetic heart valve into the native valve or previously implanted prosthetic heart valve visualizing under fluoroscopy a position of the marker to confirm a location of the commissure of the prosthetic heart valve to which the marker is attached relative to the native valve or previously implanted prosthetic heart valve.

Example 56. The method of any example herein, particularly any one of examples 52-55, wherein the marker is reflection asymmetric across an axis that is parallel to a central longitudinal axis of the frame, the central longitudinal axis extending between an inflow end and an outflow end of the frame.

Example 57. The method of any example herein, particularly any one of examples 52-56, wherein the marker is C-shaped.

Example 58. The method of any example herein, particularly any one of examples 52-55, wherein the marker is rectangular with rounded edges.

Example 59. The method of any example herein, particularly any one of examples 52-58, wherein the marker comprises tantalum.

Example 60. The method of any example herein, particularly any one of examples 52-59, wherein the marker comprises one or more apertures configured to receive a suture for fastening the marker to the commissure.

Example 61. The method of any example herein, particularly any one of examples 52-60, wherein the method is performed on a living animal or a non-living simulation.

The features described herein with regard to any example can be combined with other features described in any one or more of the other examples, unless otherwise stated. For example, any one or more of the features of one radiopaque marker can be combined with any one or more features of another radiopaque marker. As another example, any one of the radiopaque markers can be positioned at a commissure as shown for any of the other radiopaque markers.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. A prosthetic heart valve comprising:
an annular frame comprising a plurality of struts;
a plurality of leaflets arranged within the frame;
at least one commissure comprising commissure tabs of two adjacent leaflets of the plurality of leaflets connected to each other, the at least one commissure coupled to the frame; and
at least one radiopaque marker, separate from the frame, attached to the commissure, wherein the marker is configured to indicate a location of the commissure of the prosthetic heart valve.

2. The prosthetic heart valve of claim 1, wherein the marker is sewn to an exterior surface of the commissure that faces radially outward from the frame.

3. The prosthetic heart valve of claim 1, wherein the marker is sewn to a radially outward facing surface of a flexible member that is connected to the commissure tabs and forms a part of the commissure.

4. The prosthetic heart valve of claim 1, wherein the frame comprises a plurality of commissure supports, wherein each corresponding commissure support defines a commissure window in the frame, wherein at least a portion of the commissure tabs extend radially through the commissure window and are secured to and around outer surfaces of strut portions of the corresponding commissure support which define the commissure window, and wherein the commissure includes a flexible member that covers outer surfaces of the commissure tabs on an exterior of the frame.

5. The prosthetic heart valve of claim 4, wherein the marker serves as a wedge element that is disposed against outer surfaces of the commissure tabs, exterior to the commissure window and at a location where the commissure tabs separate from one another to extend around respective outer surfaces of the strut portions of the corresponding commissure support, and wherein the marker is disposed between the flexible member and the commissure tabs.

6. The prosthetic heart valve of claim 4, further comprising a wedge element secured to the commissure tabs exterior to the commissure window and at a location where the commissure tabs separate from one another to extend around respective outer surfaces of the strut portions of the corresponding commissure support, and wherein the wedge element is disposed between the commissure tabs and the flexible member.

7. The prosthetic heart valve of claim 6, wherein the marker is disposed between the wedge element and the flexible member.

8. The prosthetic heart valve of claim 6, wherein the marker is attached to an outer surface of the flexible member, the flexible member disposed between the marker and the wedge element.

9. The prosthetic heart valve of claim 1, wherein the marker is C-shaped.

10. The prosthetic heart valve of claim 1, wherein the marker is rectangular with rounded edges.

11. The prosthetic heart valve of claim 1, wherein the marker comprises tantalum.

12. A prosthetic heart valve comprising:
a frame comprising an inflow end, an outflow end, and a plurality of support posts including a commissure window;
a plurality of leaflets arranged within the frame and configured to regulate a flow of blood through the frame in one direction between the inflow end and outflow end;
at least one commissure comprising commissure tabs of two adjacent leaflets of the plurality of leaflets connected to each other and extending radially through the commissure window of a corresponding support post of the plurality of support posts, the at least one commissure connected to the corresponding support post; and
a radiopaque marker attached to the commissure, wherein the marker is configured to indicate a location of the commissure of the prosthetic heart valve.

13. The prosthetic heart valve of claim 12, wherein the marker is attached to the commissure exterior to the frame and on a radially outward facing surface of the commissure.

14. The prosthetic heart valve of claim 12, wherein the commissure comprises a wedge element disposed against the commissure tabs on an exterior of the commissure window and a flexible member covering the commissure tabs on the exterior of the commissure window, the wedge element disposed interior to the flexible member relative to a central longitudinal axis of the frame, and wherein the marker is attached to an outer surface of the flexible member.

15. The prosthetic heart valve of claim 12, wherein the commissure comprises a wedge element disposed against the commissure tabs on an exterior of the commissure window and a flexible member covering the commissure tabs on the exterior of the commissure window, the wedge element disposed interior to the flexible member relative to a central longitudinal axis of the frame, and wherein the marker is disposed between the flexible member and the wedge element.

16. The prosthetic heart valve of claim 12, wherein the commissure comprises a wedge element disposed against the commissure tabs on an exterior of the commissure window and a flexible member covering the commissure tabs on the exterior of the commissure window, the wedge element disposed interior to the flexible member relative to a central longitudinal axis of the frame, and wherein the marker is the wedge element and is disposed between the commissure tabs and the flexible member.

17. The prosthetic heart valve of claim 12, wherein the marker is reflection asymmetric across an axis that is parallel to a central longitudinal axis of the frame, the central longitudinal axis extending between the inflow end and the outflow end of the frame.

18. The prosthetic heart valve of claim 12, wherein the frame comprises a plurality of mechanical expansion and locking mechanisms that are configured to radially expand and compress the frame and that are arranged in alternating order with the plurality of support posts around a circumference of the frame.

19. A prosthetic heart valve comprising:
a frame comprising:
a plurality of support posts spaced apart about a circumference of the frame, each support post of the plurality of support posts including a commissure window; and
one or more expansion and locking mechanisms that are configured to radially expand and/or compress the frame, each of the one or more expansion and locking mechanism comprising a pair of axially aligned posts and a threaded rod extending through the pair of axially aligned posts;
a plurality of leaflets arranged within the frame and configured to regulate a flow of blood through the frame in one direction;
at least one commissure comprising commissure tabs of two adjacent leaflets of the plurality of leaflets connected to each other and extending radially through the commissure window of a corresponding support post, the at least one commissure connected to the corresponding support post; and
a radiopaque marker attached to the commissure.

20. The prosthetic heart valve of claim 19, wherein the commissure comprises a wedge element disposed against and between the commissure tabs on an exterior of the commissure window and a flexible member covering at least a portion of the commissure tabs on the exterior of the commissure window, the wedge element disposed between the flexible member and the commissure tabs, and wherein the marker is one of: attached to an outer surface of the flexible member that faces away from the frame or disposed between the flexible member and the wedge element.

21. The prosthetic heart valve of claim 19, wherein the marker is configured as a wedge element that is disposed against and between the commissure tabs on an exterior of the commissure window, and wherein the commissure comprises a flexible member covering at least a portion of the commissure tabs on the exterior of the commissure window, the marker disposed between the flexible member and the commissure tabs.

22. The prosthetic heart valve of claim 19, wherein the pair of axially aligned posts extend between an inflow end and an outflow end of the frame and are axially movable relative to one another to permit the frame to radially expand and/or compress.
